(19) United States
(12) Patent Application Publication
(10) Pub. No.: US 2020/0352143 A1
(43) Pub. Date: Nov. 12, 2020

(54) Method to Implement a CRISPR Gene Drive in Mammals

(71) Applicant: The Regents of the University of California, Oakland, CA (US)

(72) Inventors: Kimberly Cooper, San Diego, CA (US); Ethan Bier, San Diego, CA (US); Hannah Grunwald, San Diego, CA (US); Valentino Gantz, San Diego, CA (US); Gunnar Poplawski, Loerrach (DE)

(21) Appl. No.: 16/960,691

(22) PCT Filed: Jan. 10, 2019
(86) PCT No.: PCT/US2019/013011

§ 371 Date: Jul. 8, 2020

Related U.S. Application Data
(60) Provisional application No. 62/615,727, filed on Jan. 10, 2018.
(60) Provisional application No. 62/668,966, filed on May 9, 2018.

(51) Int. Cl. A01K 67/027; C12N 15/11; C12N 9/22

(52) U.S. Cl. CPC A01K 67/0275; C12N 15/111; C12N 9/22; C12Y 114/18001; A01K 2227/105; A01K 2267/0393; C12N 2310/20; A01K 2217/072

(57) ABSTRACT

Provided are systems, constructs, genetically modified organisms, and methods for creating transgenic rodent research and commercial models of human physiology, disease, syndromes, and disorders. Provided are genetically modified rodents encoding for an sgRNA useful in a Cas9-mediated split gene-drive system for optimization of the gene drive system in rodents.

### CROSS REFERENCE TO RELATED APPLICATIONS

This application claims the priority benefit of U.S. Provisional Application Nos. 62/615,727 and 62/668,966, filed Jan. 10, 2018 and May 9, 2018, respectively, which applications are incorporated herein by reference.

### GOVERNMENT SPONSORSHIP

This invention was made with government support under grant No. R21GM129448 by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jan. 10, 2019, is named 24978-0472_SL.txt and is 10,636 bytes in size.

### BACKGROUND

Currently, alleles at multiple loci in the mouse genome must be combined by Mendelian genetics in crosses of animals to one another to produce a desired compound mutant genotype. For example, to combine homozygous mutations at two loci, animals that are heterozygous for each gene must be produced by breeding, and these are subsequently crossed to one another. The frequency of homozygosity for each allele is 1:4 the frequency of homozygosity for both genes is 1:16. Further, the average litter of mice is approximately 10 pups, and the generation time from conception to reproductive age is about 3 months. Therefore this method requires a substantial number of animals and time. With the addition of each new locus (three, four, etc.), the cost measured in animals, time, and money increases exponentially. These factors increase substantially more if two or more loci are genetically linked, which requires rare recombination events to combine engineered alleles on the same chromosome.

Consequently, there remains a need for better and cheaper methods for development of research and commercial animal models of human physiology and disease.

In the mosquito and fly, Cas9 expression was limited to the germ line by use of the Vasa promoter. One purpose of sexual reproduction is to “shuffle the deck” by recombining the maternal and paternal genomes at each generation. Given the prevalence of double strand break (DSB) formation during meiotic recombination, an active NHEJ pathway would be highly mutagenic. Indeed, the molecular mechanisms of non-homologous end joining (NHEJ) are repressed during meiosis in many species, including mice, and homology directed repair (HDR) occurs by inter-homologue rather than inter-sister exchange. However, the frequency of inter-homologue recombination after CRISPR-Cas9 induced DSB formation in the germ line has not yet been measured in a mammal. While CRISPR-Cas9 gene drives have been implemented in two species of insects, flies, and mosquitoes, it has not been reported in any non-insect animal species.

### SUMMARY OF THE INVENTION

A CRISPR-Cas9 mediated gene drive leverages the native cellular mechanism of homology directed repair to copy a desired allele from one chromosome to another. This process can convert a heterozygous genotype to homozygosity in a single generation of any animal, including mammals such as rodents.

This disclosure provides a new paradigm for development of research and commercial animal models of human physiology and disease as well as for rodent population suppression. In embodiments, the present invention utilizes CRISPR-Cas9 gene drives to facilitate rodent husbandry while lowering production costs and time when compared to using Mendelian genetics to produce desired mutant genotypes. The invention provides a research tool by producing animal models of human physiology and disease, which can be implemented in a wide variety of applications to model disease, test drug efficacy, and metabolism.

In embodiments, the present invention utilizes CRISPR-Cas9 gene drives to facilitate rodent husbandry to produce desired mutant genotypes, which can be used to control wild rodent populations. In embodiments, mutant genotypes enhancing female or male sterility can be produced as part of a rodent population suppression strategy. In embodiments, the invention uses the split gene-drive system to transmit a transgene encoding genes such as the Sry gene to all, or nearly all, offspring, thus rendering all such progeny male. Furthermore, in embodiments, the system can render any animals, such as rodents, that escaped conversion sterile and/or sensitive to new pesticides specific to rodents or to pesticides to which the existing population had acquired resistance.

In embodiments, the present invention provides a gene drive “reporter” mouse (Tyrosinase^{CopyCat}) that can facilitate optimization of the gene drive in various contexts. This mouse encodes an sgRNA in exon 4 of the tyrosinase gene, but unlike an insect Mutagenic Chain Reaction System, it does not also encode the Cas9 gene. Consequently this gene drive element is not able to copy itself autonomously and instead requires an exogenous source of Cas9. In embodiments, this reporter mouse can be used to improve the efficiency by altering the developmental timing and cell type specificity of Cas9 expression and by testing modified versions of the Cas9 enzyme.

In embodiments, the invention provides that two separate genetic elements comprise the split trans-complementing gene-drive system in which the first element (A) carries the one or more desired alleles at a defined autosomal location such that it can be driven by a Cas9 source provided in trans (element B). The A element can also carry several guide RNAs (gRNAs): 1) a gRNA driving the element A at its insertion site, 2) a gRNA driving the element B at its insertion site, and 3) multiple gRNAs targeting coding sequences of several genes required for mutagenesis through non-homologous end joining (NHEJ). When strains A and B are crossed, however, the Cas9 carried by element B drives copying of both element A and element B at their respective locations by means of copying them onto the homologous chromosome, the resulting progeny carrying both elements contain the one or more desired alleles, and capable of transmitting these alleles on to nearly all their progeny and subsequent generations.

In alternative embodiments, the invention can include the gRNA driving element B along with the Cas9 source to create a full gene drive at the locus. The advantage of this latter configuration is that it reduces the number of gRNAs needed to be expressed from element A. The advantage of the former trans-complementing MCR configuration is that both strains A and B would be non-driving, simplifying husbandry of these strains prior to crossing them to establish a bipartite gene drive. Elements A and B or the corresponding genomic insertion sites on wild-type chromosomes can also carry fluorescent marker genes to distinguish transgenic from wild-type chromosomes.

In embodiments, the present invention combines two concepts: 1) the split or trans-complementing mutagenic chain reaction (MCR) form of gene drive, and 2) the fact that many human diseases, syndromes, and disorders are the effect of chromosomal deletions or translocations that eliminate function of multiple genes (e.g. Williams-Beuren Syndrome, which deletes approximately 28 genes). In embodiments, the invention uses the split gene-drive system and by encoding clusters of gRNAs that target subsets of the genes of a specific disease, syndrome, or disorder to multiplex compound knockout alleles to assess multigenic phenotypes. In embodiments, the invention inserts genetically encoded elements in any locus in the genome. In embodiments, alleles encoded with the sgRNAs are made that insert exogenous components of a novel biosynthetic pathway into the rodent genome. Resulting engineered rodents may produce compounds not present in wild type animals. In embodiments, the invention humanizes one or more genes of the rodent genome, alone or in combination, by inserting genes from the human genome to replace the homologous rodent counterparts. Resulting engineered rodents make the rodent a better model (research tool) for disease and drug development. In embodiments, the invention mutates genes of the rodent to replicate genetically complex human diseases that require changes at multiple loci.

### BRIEF DESCRIPTION OF THE DRAWINGS

A better understanding of the features and advantages of the present disclosure will be obtained by reference to the following detailed description that sets forth illustrative embodiments, in which the principles of the disclosure are utilized, and the accompanying drawings of which:

FIG. 1 depicts the Tyrosinase Exon 4 “CopyCat” transgene that was inserted into the mouse genome by homologous recombination. As a visible marker of inheritance, the knock in allele carries a CMV Enhancer and Promoter-driven mCherry transgene with a bovine growth hormone polyadenylation signal (bGH poly[A]). A Human U6 Promoter (and downstream sequence) controls the transcription of a gRNA (TyrEx4-gRNA1) that targets the homologous location of insertion into the target wild type locus in heterozygous animals.

FIGS. 2A-2F depict the sample genotype results for each allele using primers that are indicated in Table 3. For all panels: dark blue arrows indicate the wild type alleles or internal positive controls (IPC, amplifies interleukin2 on chromosome 3), light blue arrows indicate transgenes. Red arrows denote relevant size markers in the DNA ladder for comparison. FIG. 2A shows genotyping for constitutive H11:Cas9 (HCC) and H11:LoxSTOPLox Cas9 (HLC). Right-HCC: Band at 425 bp indicates the Cas9 transgene. The band at 200 bp indicates the wild type (non-transgenic) H11 allele. Left-HLC: The band at 350 bp indicates the LoxSTOPLox Cas9 transgene. The band at 200 bp indicates wild type (non-transgenic) H11 allele. FIG. 2B shows genotyping for constitutive Rosa26:Cas9 (RCC) or Rosa26:LoxSTOPLoxCas9 (RLC). The band at 1.2 kb indicates wild type (non-transgenic) Rosa26 allele. The bands at 220 bp indicate each respective Cas9 transgene. FIG. 2C shows genotyping for Vasa: Cre and Stra8: Cre. This genotyping strategy identifies presence or absence of the Cre transgene but not copy number. In both cases, an internal positive control at 324 bp (IPC) confirms successful amplification. Left: Vasa:Cre primers only amplify the Vasa:Cre transgene. Right: Stra8: Cre primers only amplify the Stra8: Cre transgene. Each primer pair contains a primer specific to the regulatory sequence and a primer within Cre. FIG. 2D shows genotyping for Tyr^{Chinchilla}. The Tyr^{Chinchilla} primers flank the SNP and therefore amplify a 392 bp product regardless of Tyr^{Chinchilla} genotype. This amplicon was purified and sequenced to reveal the genotype as in FIG. 5. FIG. 2E shows genotyping to determine presence of Tyr^{CopyCat} transgene. This strategy identifies presence or absence of the transgene but not copy number. An internal positive control at 324 bp (IPC) confirms successful amplification. The band at 838 bp indicates the presence of the Tyr^{CopyCat} transgene in animals that inherit the original Tyr^{CopyCat} chromosome and also in animals that copy the Tyr^{CopyCat} allele to the Tyr^{Chinchilla} marked chromosome by HDR. FIG. 2F shows gGenotyping to amplify Tyr exon 4 which can include the Tyr^{CopyCat} transgene. The band at 2606 bp is an amplicon that includes the Tyr^{CopyCat} transgene. The band at approximately 400 bp is the non-transgenic allele that was purified and sequenced to reveal NHEJ indels in individuals that inherited Cas9 and did not repair DSBs by HDR.

FIGS. 3A-3B. FIG. 3A depicts a schematic of 1.75 kb Tyr^{CopyCat} knock-in allele. FIG. 3A discloses SEQ ID NO: 29. FIG. 3B depicts the Cas9 cleavage, where the encoded sgRNA targets Cas9 cleavage of the homologous chromosome precisely at the point of Tyr^{CopyCat} insertion. The underlined sequence corresponds to the PAM site of the sgRNA recognition sequence, and asterisks in the lower sequence denote the predicted site of DSB formation (SEQ ID NO: 30). Mice with two null Tyr alleles will be albino. “Ch” represents the tightly linked chinchilla allele (Tyr^{Ch}) in exon 5 that allows for the tracking of inheritance of the homologous target chromosome.

FIG. 4 depicts the breeding scheme used to test the efficiency of DSB and inter-homologue recombination with constitutive Cas9 transgenes.

FIG. 5 depicts Sanger sequencing traces of Tyrosinase exon 5 differentiated individuals that were wild type, heterozygous, and homozygous for the Chinchilla SNP (SEQ ID NOS 31-33 and 31, respectively, in order of appearance).

FIGS. 6A-6F depict embryonic Cas9 activity does not copy the Tyrosinase^{CopyCat} allele to the target chromosome. FIG. 6A shows knock-in strategy using the Tyr^{CopyCat} targeting vector. The U6-Tyr4a gRNA and CMV-mCherry were inserted by HDR into the cut site of the Tyr4a gRNA. FIG. 6B shows the genetically encoded Tyr^{CopyCat} element, when combined with a transgenic source of Cas9 is expected to induce a DSB in the TyrChinchilla-marked target chromosome, which could be repaired by inter-homologue HDR. FIG. 6C shows breeding strategy to unite Tyr^{CopyCat} with a constitutive Cas9 transgene followed by test cross to Tyr^{Null}. FIG. 6D shows the quantification of F3 test cross offspring. FIG. 6E shows a representative Rosa26-Cas9 F2 litter. Black mice did not inherit Tyr^{CopyCat}. Grey mice inherited Tyr^{CopyCat} but not Cas9. White mice inherited both transgenes. FIG. 6F shows a representative litter in which all inherited H11-Cas9. The mosaic mice also inherited Tyr^{CopyCat}.

FIGS. 7A-7D show mCherry fluorescence marks Tyrosinase^{CopyCat} tails and ears. (FIGS. 7A and 7B) Two tail tips from F2 mice of the Rosa26:Cas9 lineage with Tyr^{CopyCat} (left and middle) and one from a mouse that did not inherit the Tyr^{CopyCat} transgene (right; Tyr^{WT/Ch}) mCherry is visible only in tails with an allele of the Tyr^{CopyCat} transgene. (FIGS. 7C and 7D) F3 offspring of the constitutive Rosa26:Cas9 lineage. The left mouse inherited the original Tyr^{CopyCat} transgene with mCherry fluorescence in an outcross to CD-1 Tyrosinase^{Null}. The left mouse inherited the Tyr^{Ch}-marked target chromosome with an NHEJ mutation and no mCherry fluorescence.

FIGS. 8A-8B show Cas9 activity in the female germline copies the TyrosinaseCopyCat allele to the target chromosome. FIG. 8A shows the breeding strategy to produce Tyr^{CopyCat/Chinchilla} mice with a conditional Cas9 transgene and a germline restricted Cre transgene. F3 offspring were test crossed to TyrNull animals to assess F4 phenotypes and genotypes. FIG. 8B shows the quantification of the efficiency of HDR conversion in F4 test cross offspring.

FIG. 9 shows genotype conversion by an active genetic element was observed in the female germline and not in the male germline or in the early embryo. Schematic representation of early embryonic and male and female germline development. Differences in germline specification coincide with presence or absence of observed HDR. [PGCs: primordial germ cells, n: number of homologous chromosomes, c: chromosome copy number. Asterisk indicates the difference between male sperm (n, 1c) and female ovum, which remains (n, 2c) until second polar body extrusion after fertilization.]

### DETAILED DESCRIPTION

All publications, patents, and patent applications mentioned in this specification are herein incorporated by reference to the same extent as if each individual publication, patent, or patent application was specifically and individually indicated to be incorporated by reference.

It is understood that aspects and embodiments of the invention described herein include “consisting” and/or “consisting essentially of” aspects and embodiments. Other objects, advantages and features of the present invention will become apparent from the following specification taken in conjunction with the accompanying figures.

### Definitions

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “CopyCat element” refers to a split Cas protein and gRNA configuration, in which only the gRNA can be inserted at the cut site. A CopyCat element can refer to the self-propagating gRNA. The Cas9 source can be supplied in trans, allowing the CopyCat element to be segregated away from the Cas9 source as desired, at which point it will obey the laws of standard Mendelian inheritance. In the presence of Cas9, however, the CopyCat element can be actively copied to its sister chromosome, resulting in it becoming homozygous. An advantage of the CopyCat element is that one can segregate the source of Cas9 away from the CopyCat element and then manipulate such element via standard Mendelian genetics.

The term “endonuclease” refers to an enzyme that cleaves the phosphodiester bond within a polynucleotide chain. Endonucleases can include Cas proteins, such as Cas9.

The term “guide polynucleotide” refers to a polynucleotide sequence that can form a complex with an endonuclease (e.g., Cas protein such as Cas9) and enables the endonuclease to recognize and optionally cleave a target site on a polynucleotide such as DNA. That is, a guide polynucleotide is any polynucleotide sequence having sufficient complementarity with a target polynucleotide sequence to hybridize with the target sequence and direct sequence-specific binding of a CRISPR complex to the target sequence. A guide polynucleotide that solely comprises ribonucleic acids is also referred to as a “guide RNA” or “gRNA”. Synthetic guide RNA is referred to as “sgRNA”. gRNA and sgRNA can be utilized interchangeably.

The term “effector cassette” can refer to a genetic construct including a transgene encoding a protein that when expressed exerts a desired effect (e.g., a trans-complementing, functional or reporter gene, such as Tyrosinase, or portion thereof, to affect melamin biosynthesis, etc.).

The term “active genetics” refers to genetic manipulations in which Cas9 and gRNA elements are used to copy a genetic element from one chromosome to the identical insertion site on the sister chromosome and/or actively edit a genome sequence (e.g. sequence deletions, additions) by single-unit MCR or trans-complementing MCR.

The term “genetic drive” can refer to the inheritance of an allele of a diploid gene more than 50% of the time (i.e., more than by random chance alone).

The term “trans-complementing MCR” refers to a configuration in which a gRNA bearing transgene not encoding Cas9 is combined with a Cas9 bearing transgene to actively copy the gRNA bearing transgene to its sister chromosome, actively copy the Cas9 bearing transgene to its sister chromosome, and/or actively edit the genome sequence.

The term “trans-complementing MCR construct,” “transgenic element,” “trans-complementing MCR element,” and the like, refers to a construct that, when co-expressed with at least one other trans-complementing MCR construct, results in trans-complementing MCR. A trans-complementing MCR construct can comprise sequences encoding Cas9, gRNAs, and/or effector cassettes.

The term “rodent,” and the like refers to mammals of the order Rodentia and includes, but not limited to, all species of mice, rats, squirrels, prairie dogs, porcupines, beavers, guinea pigs, hamsters, gerbils, and capybara.

### CRISPR/Cas System

The present disclosure is based in part on the CRISPR/Cas system, a genome editing tool that can be used in a wide variety of organisms (e.g., used to add, disrupt, or change the sequence of specific genes). The CRISPR/Cas9 system is based on two elements. The first element, Cas9, is an endonuclease that has a binding site for the second element, which is the guide polynucleotide (e.g., guide RNA). The guide polynucleotide (e.g., guide RNA) directs the Cas9 protein to double stranded DNA templates based on sequence homology. The Cas9 protein then cleaves that DNA template. By delivering the Cas9 protein and appropriate guide polynucleotides (e.g., guide RNAs) into a cell, the organism's genome is cut at a desired location. Following cleavage of a targeted genomic sequence by a Cas9/gRNA complex, one of two alternative DNA repair mechanisms can restore chromosomal integrity: 1) non-homologous end joining (NHEJ) which generates insertions and/or deletions of a few base-pairs (bp) of DNA at the gRNA cut site, or 2) homology-directed repair (HDR) which can correct the lesion via an additional “bridging” DNA template that spans the gRNA cut site. Further aspects of the CRISPR/Cas system known to those of ordinary skill are described in PCT Publication No. WO 2017/049266, the entire contents of which are hereby incorporated by reference.

### Autocatalytic Genome Editing Using Trans-Complementation

The present disclosure provides methods and compositions for autocatalytic genome editing based on genomic integration of split or trans-complementing Mutagenic Chain Reaction (MCR) constructs. Trans-complementing MCR provides a split system, which can consist of two separate transgenic elements which when combined can lead to autocatalytic copying of elements to sister chromosomes and/or active genome sequence editing. One element expresses a Cas9 endonuclease (i.e. the Cas9 bearing element) and the other element (i.e. the non-Cas9 bearing element), which can be inserted elsewhere on the same chromosome as the Cas9-bearing element or on a different chromosome, encodes at least one gRNA that can cut at the site of genomic insertion of the non-Cas9 bearing element (i.e., gRNA1). A second gRNA that cuts at the genomic site of insertion of the Cas9 bearing element can be encoded in either element (i.e., gRNA2). Furthermore, many human diseases, syndromes, and disorders are the effect of chromosomal deletions or translocations that eliminate function of multiple genes (e.g. Williams-Beuren Syndrome, which deletes approximately 28 genes). By encoding clusters of gRNAs that target subsets of these genes, it is possible to multiplex compound knockout alleles to assess multigenic phenotypes. When these two elements are carried in the same individual (e.g., in progeny resulting from a cross of two individuals carrying a respective one of the two elements) both elements are actively copied onto their sister chromosomes and any additional gRNAs cause active genome sequence editing.

In embodiments, a trans-complementing MCR described herein can mitigate problems associated with single-unit MCR since the two separate elements (i.e. Cas9 and gRNA1) can each be propagated safely as neither alone can create a gene-drive. Also, neither element alone can create a significant level of off-target mutagenesis since both elements must be combined. Thus, the two separate components of the trans-complementing MCR can be kept separate until the time they are to be used at which point the two stocks can be crossed. The resulting progeny of this cross can then carry both elements which can propagate as a unit like a single-unit MCR.

A trans-complementing MCR can have the same high efficiency observed for a single-unit MCR (e.g., one in which the Cas9 source and a gRNA are carried as a single cassette inserted into the site cut by the gRNA=95% conversion efficiency).

Trans-complementing MCR generally requires at least two trans-complementing constructs, although there could be more. The first trans-complementing construct, which can be referred to as the Cas9 bearing construct, comprises: (1) a DNA fragment encoding an endonuclease (e.g. Cas9 protein) or homolog that directs its expression in the germline cells, and (2) optionally, a sequence encoding a guide polynucleotide (e.g., guide RNA) that can cut at the site of genomic insertion of the first trans-complementing construct (i.e., gRNA2). The second trans-complementing construct, which can be referred to as the non-Cas9 bearing construct, comprises: (1) one or more sequences encoding one or more guide polynucleotides (e.g., guide RNAs); and (2) one or more effector cassettes (e.g., a DNA sequence that carries out a function). The one or more sequences encoding one or more guide polynucleotides in the second trans-complementing construct can include: (1) a sequence encoding a guide polynucleotide that can cut at the site of genomic insertion of the second trans-complementing construct (i.e., gRNA1), (2) optionally, a sequence encoding a guide polynucleotide that can cut at the site of genomic insertion of the first trans-complementing construct (i.e., gRNA2), and (3) optionally one or more sequences encoding guide polynucleotide(s) that can cut at loci required for generating research models of human physiology and diseases and/or syndromes (e.g., gRNA3, gRNA4, gRNA5, etc.). The trans-complementing constructs or proteins encoded therein can also include functional groups, such as for example a GFP domain or other fluorescent marker, for visualization purposes.

Each of the first and second trans-complementing constructs can be inserted into the genome independently (e.g., by co-injecting a plasmid containing the first trans-complementing construct with a plasmid encoding only the gRNA2 transcript (if needed), and by injecting a plasmid containing the second trans-complementing construct with a plasmid encoding Cas9 or purified Cas9 protein). For example, a plasmid encoded cassette carrying genes encoding the Cas9 protein flanked by homology arms corresponding to the genomic sequences straddling the target site injected with a plasmid encoding only the gRNA2 transcript results in cleavage and homology driven insertion of the sequence encoding the Cas9 protein element into the targeted locus. In another example, a plasmid encoded cassette carrying genes encoding guide RNA(s) targeting genomic sequences of interest and/or an effector cassette, both of which are flanked by homology arms corresponding to the genomic sequences straddling the target site, injected with a plasmid encoding Cas9 or purified Cas9 protein results in cleavage and homology driven insertion of the sequence encoding the guide RNA(s) targeting genomic sequences of interest and/or an effector cassette into the targeted locus.

In embodiments where the sequence encoding the gRNA that cuts at the Cas9 bearing construct site (i.e., gRNA2) is included in the non-Cas9 bearing construct (i.e. the second trans-complementing construct), each of the first and second trans-complementing constructs, if integrated into the genome of germline cells at their respective gRNA sites, can be inherited in a standard Mendelian fashion. When individuals separately carrying these two elements are crossed to each other, the resulting progeny can have both elements and the two elements can propagate like a standard MCR element in that the two parts (i.e., the Cas9 bearing construct inserted at gRNA2's cut-site, and the non-Cas9 bearing construct inserted at gRNA1's cut-site) can copy themselves from one chromosome to the sister chromosome. Because both elements can copy themselves onto the opposing chromosome, these progeny become homozygous for the constructs and all (or nearly all) of the progeny's progeny can inherit the constructs. Also, any additional gRNAs present create homozygous mutations at their respective cut sites by active genome sequence editing and any effector constructs become homozygous as well. The progeny's progeny themselves become homozygous via trans-complementing MCR, and thus can pass on both constructs to their offspring. Thus, trans-complementing MCR generates homozygous mutant phenotypes in a single generation.

In embodiments where the sequence encoding the gRNA that cuts at the Cas9 bearing construct site (i.e., gRNA2) is included in the Cas9 bearing construct (i.e. the first trans-complementing construct), the first trans-complementing construct can always copy itself onto the opposing chromosome and all (or nearly all) progeny from such a parent inherit the first trans-complementing construct. The second trans-complementing construct, however, can be inherited in a standard Mendelian fashion. When individuals separately carrying the first and second trans-complementing constructs are crossed to each other, the resulting progeny can have both constructs and the second trans-complementing construct (i.e. the non-Cas9 bearing construct) can then propagate like a standard MCR element in that the second trans-complementing construct (inserted at gRNA1's cut-site) can copy itself from one chromosome to the sister chromosome. Because both elements can copy themselves onto the opposing chromosome, these progeny become homozygous for the constructs and all (or nearly all) of the progeny's progeny can inherit the constructs. Also, any additional gRNAs present create homozygous mutations at their respective cut sites by active genome sequence editing and any effector constructs become homozygous as well. The progeny's progeny themselves become homozygous via trans-complementing MCR, and thus can pass on both constructs to their offspring. Thus, trans-complementing MCR generates homozygous mutant phenotypes in a single generation.

In embodiments, the disclosure provides methods of independently inserting a first trans-complementing construct into the germline of a first organism (e.g. rodent) and a second trans-complementing construct into the germline of a second organism (e.g. rodent), and obtaining transgenic organisms carrying the insertion of either one of the constructs on one copy of a chromosome. In embodiments, mating between one organism having a first trans-complementing construct and a second organism having a second trans-complementing construct yields progeny containing both constructs, which results in each construct spreading to both chromosomes to create homozygous mutations for each construct by trans-complementing MCR. Any additional gRNAs present create homozygous mutations at their respective cut sites by active genome sequence editing and any effector constructs become homozygous as well. A transgenic organism containing both constructs propagates mutations via the germline to its offspring with greater than 95% efficiency.

In embodiments, trans-complementing MCR can be used to accelerate genetic manipulations and genome engineering. For example, an active trans-complementing MCR drive may provide faster propagation of a genetic trait than passive Mendelian inheritance. In some embodiments, trans-complementing MCR can selectively add, delete, or mutate genes. In some embodiments, trans-complementing MCR can form a gene drive for spreading genes or exogenous DNA fragments through a population of an organism (e.g. a rodent) to combat the organism and any diseases or pathogens carried by it (e.g. mutating genes to confer infertility or increased susceptibility to pesticides). That is, trans-complementing MCR can be used to disperse (or drive) transgenes into rodent populations to selectively inhibit propagation of pest populations and combat propagation of rodent borne pathogens or diseases. In other embodiments, trans-complementing MCR can form a gene drive for spreading genes or exogenous DNA fragments through a population of an organism (e.g. a rodent) to develop research and/or commercial models of human physiology and diseases or syndromes (e.g. mutating genes to confer specific chromosomal additions, deletions, or translocations associated with diseases and syndromes).

In some embodiments, the present disclosure provides trans-complementing MCR drives which offer potential husbandry advantages. In embodiments, there are two separate trans-complementing drives for the cas9 <cas9> and gRNAs <gRNA1; gRNA2; gRNA3; effector cassette> wherein gRNA1 cuts at the integration site of the <gRNA1; gRNA2; gRNA3; effector cassette> element and gRNA2 directs cleavage at the site of cas9 genomic insertion. Since neither of the two constructs alone constitutes a drive, each single element can be propagated safely as a separate stock. When the two stocks are crossed (possibly after amplification of each of the stocks for release purposes), a full drive can result. In progeny of this cross, the resulting <cas9> and <gRNA1; gRNA2; gRNA3; effector cassette> can combine to create a drive that can behave thereafter as a linked <cas9; gRNA1; gRNA2; gRNA3; effector cassette> MCR.

In some embodiments, the present disclosure provides alternative trans-complementing MCR drives which offer potential husbandry advantages. In embodiments, there are two separate trans-complementing drives for the cas9 <cas9; gRNA2> and gRNAs <gRNA1; gRNA3; effector cassette> wherein gRNA1 cuts at the integration site of the <gRNA1; gRNA3; effector cassette> element and gRNA2 directs cleavage at the site of <cas9; gRNA2> genomic insertion. The <cas9; gRNA2> construct behaves like a full gene drive. However, the <gRNA1; gRNA3; effector cassette> construct alone does not constitute a gene drive and can be propagated safely as a separate stock. When the two stocks are crossed (possibly after amplification of each of the stocks for release purposes) a full drive for both elements can result. In progeny of this cross the resulting <cas9; gRNA2>; <gRNA1; gRNA3; effector cassette> can combine to create a drive that can behave thereafter as a linked <cas9; gRNA1; gRNA2; gRNA3; effector cassette> MCR.

Methods of the disclosure can be used to generate specific strains, breeds, or mutants of an organism; for one-step mutagenesis schemes to generate scoreable recessive mutant phenotypes in a single generation; facilitate basic genetic manipulations in organisms; and accelerate genetic manipulations in organisms.

In embodiments, DNA cuts generated by an endonuclease such as Cas9 may be corrected using different cellular repair mechanisms, including error-prone non-homologous end joining (NHEJ) and Homology Directed Repair (HDR). In some embodiments, a trans-complementing element is integrated into a genome using HDR. Trans-complementing elements are often integrated into a genome using homology directed repair (˜90-100% efficiency).

Trans-complementing elements, when combined, can form an active gene drive and the efficiency of a trans-complementing element integrating into a genome is about 80%, 85%, 90%, 91%, 92%, 93%, 94%, 95%, 96%, 97%, 98%, 99%, 99.5%, 99.9%, or more than 99.9%. In embodiments, the efficiency of allelic conversion of a trans-complementing element in an active gene drive into a genome is about 80%, 85%, 90%, 91%, 92%, 93%, 94%, 95%, 96%, 97%, 98%, 99%, 99.5%, 99.9%, or more than 99.9%. Trans-complementing elements, when combined, form an active gene drive and nearly double their frequency in a population at each generation, as they convert non-MCR chromosomes derived from parents to the MCR condition. This results in a potent gene drive system for spreading genes or exogenous DNA fragments throughout populations of animal organisms such as mammals, and including rodents.

In embodiments, a trans-complementing construct is integrated into a defined site on a single copy of a chromosome. For instance, specific targeting via a guide polynucleotide (e.g., gRNA or sgRNA) directs an endonuclease (e.g., Cas9) to cleave the genome at a specific site, and the trans-complementing construct is inserted into the site by homologous repair. The trans-complementing construct in combination with a supporting trans-complementing construct carry all the elements necessary for insertion of the trans-complementing construct into the same site on a second copy of the chromosome, and combined the trans-complementing constructs cleave the other allele in a cell at the same place as the trans-complementing construct and insert the trans-complementing construct into the second copy of the chromosome thereby resulting in the insertion becoming homozygous. The MCR insertion becomes homozygous in the germline, resulting in progeny of an individual carrying an MCR allele inheriting it. The mutation spreads from a single chromosome to both chromosomes in the next generation to once again become homozygous.

In embodiments, an autocatalytic genetic behavior with self-propagating genetic elements can be achieved in which mutants are generated by two co-expressed trans-complementing constructs that combined encode at least the following two components: (1) a Cas9 protein; and (2) gRNAs targeted to genomic sequences of interest. Such a system can result in Cas9 cutting genomic targets at the sites determined by the gRNAs followed by insertion of a Cas9 bearing element and a non-Cas9 bearing element (e.g. gRNA/effector sequence-bearing element) into the respective loci via HDR. Expression of Cas9 and the gRNAs from the insertion alleles can then lead to cleavage of the opposing alleles followed by HDR-driven insertion of the respective Cas9/gRNA elements into the companion chromosomes.

In embodiments, methods for autocatalytic genome editing in an organism are provided, the methods comprising: (1) integrating a first transgenic element comprising a gene for an endonuclease and optionally a sequence for a guide polynucleotide engineered to target an integration site of the first transgenic element into a first organism; (2) integrating a second transgenic element comprising a sequence for a guide polynucleotide engineered to target an integration site of the second transgenic element, optionally a sequence for a guide polynucleotide engineered to target an integration site of the first transgenic element, one or more sequences for one or more guide polynucleotides engineered to target loci associated with specific diseases or syndromes and cause site directed mutagenesis, and one or more effector cassettes into a second organism; and (3) crossing the first and second organism, wherein crossing the first and second organisms produces progeny that propagates the first transgenic element, the second transgenic element, and site directed mutations to target loci by mutagenic chain reaction to produce research models for diseases and syndromes. In embodiments, the organism is a rodent. In embodiments, the endonuclease is Cas9. In embodiments, the first transgenic element comprises a guide polynucleotide engineered to target an integration site of the first transgenic element. In embodiments, the second transgenic element comprises a guide polynucleotide engineered to target an integration site of the first transgenic element. In embodiments, the one or more effector cassettes comprise the specific loci associated with specific diseases and/or syndromes.

For many diseases, syndromes, and disorders the genes involved are known to those of ordinary skill and can be targets for site directed mutations by mutagenic chain reaction. As an example; Williams-Beuren Syndrome includes deletion of the genes of chromosome 7q11.23 which spans approximately 28 genes.

In embodiments, the present invention provides genetically modified rodents having a Cas9-mediated split gene-drive system for creating transgenic rodents capable of mimicking human physiology and diseases, syndromes, or disorders. In embodiments, the genetically modified rodents further have a Cas9-mediated gene drive system targeting fertility and gender loci. In addition, in embodiments, the present invention provides that gRNAs direct Cas9 cleavage of pesticide-resistance loci, or direct insertion of new loci conferring a new pesticide sensitivity, thereby rendering the rodents sensitive to pesticides.

In embodiments, the present invention provides systems, constructs, genetically modified organisms for a more efficient development of research tools for human physiology and diseases, syndromes, or disorders. In embodiments, the present invention also provides methods for reducing or eliminating local populations of rodents, and associated diseases.

### EXAMPLES

### Cloning of the Tyrosinase^{CopyCat} Transgene

All primers for cloning are listed in Table 1.

TABLE 1
Primers that were used for cloning
the Tyrosinase^{CopyCat} transgene.

| | | SEQ ID |
|---|---|---|
| | | NO: |
| V851 | CCAGCTAGCAGAGGGCCTATTTCCC | 1 |
| V852 | GAGCTCGAATTCACTGGCCGTC | 2 |
| V853 | TAGGCCCTCTGCTAGCTGGGACATTGATTATTGACTAG | 3 |
| | TTATTAATAGTAATCAATTACG | |
| V854 | TGTAAAACGACGGCCAGTGAATTCGAGCTCCCATAGAG | 4 |
| | CCCACCGCAT | |
| V875 | GTTATGGCCGATAGGTGCATGTTTTAGAGCTAGAAATA | 5 |
| | GCAAGTTAAAATAAGG | |
| V878 | GGTGTTTCGTCCTTTCCACAAG | 6 |

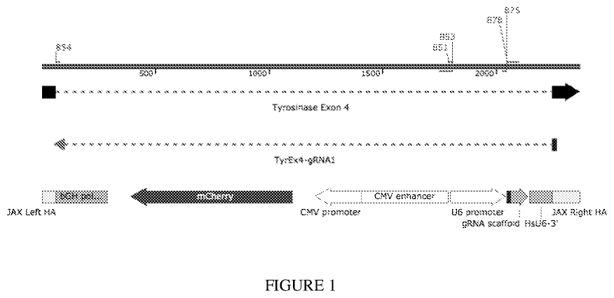

Using primers v851 and v852 a backbone for bacterial propagation that also contained a Human U6 promoter and gRNA scaffold was amplified. A second fragment of DNA that contained the CMV enhancer and promoter driving expression of the mCherry fluorophore from plasmid #548 (provided by Dr. Mark Tuszynski) was amplified, using the primers v853 and v854 (Table 1). The two fragments were joined using the Gibson Assembly technique with reagents from New England Biolabs (NEB) (Cat. #E5520S) to obtain the plasmid pVG211, which carried all the components of the CopyCat except for the gRNA target sequence. To obtain the final transgene sequence, the Tyrosinase Exon 4 gRNA target (TyrEx4-gRNA1) sequence was inserted by performing a plasmid primer mutagenesis using the primers v878 and v875 and the NEB Q5 Site-Directed Mutagenesis Kit (Cat. #E0554S) to obtain the pVG242 plasmid. This plasmid was modified to include homology arms for homologous recombination into the Tyrosinase locus, precisely at the TyrEx4-gRNA1 target cut site. This targeting construct was then used for mouse transgenesis by pronuclear injection followed by screening for germline transmission in the progeny of a backcross. The resulting inserted transgene is represented in FIG. 1.

### CopyCat Transgene Sequence

Below is displayed the sequence of the transgene in bold, inserted into the mouse genome (underlined sequences). Tyrosinase CopyCat Exon 4:

(SEQ ID NO: 7)
TATTTTTGAACAATGGCTGCGAAGGCACCGCCCTCTTTTGGAAGTTTAC
CCAGAAGCCAATGCCATAGAGCCCACCGCATCCCCAGCATGCCTGCTAT
TGTCTTCCCAATCCTCCCCCTTGCTGTCCTGCCCCACCCCACCCCCCAG
AATAGAATGACACCTACTCAGACAATGCGATGCAATTTCCTCATTTTAT
TAGGAAAGGACAGTGGGAGTGGCACCTTCCAGGGTCAAGGAAGGCACGG
GGGAGGGGCAAACAACAGATGGCTGGCAACTAGAAGGCACAGTCGAGGC
TGATCAGCGAGCTCTAGCATTTAGGTGACACTATAGAATAGGGCCCTCT
AGATGCATGCTCGAGCGGCCGCCAGTGTGATGGATATCTGCAGAATTCT
TACTTGTACAGCTCGTCCATGCCGCCGGTGGAGTGGCGGCCCTCGGCGC
GTTCGTACTGTTCCACGATGGTGTAGTCCTCGTTGTGGGAGGTGATGTC
CAACTTGATGTTGACGTTGTAGGCGCCGGGCAGCTGCACGGGCTTCTTG
GCCTTGTAGGTGGTCTTGACCTCAGCGTCGTAGTGGCCGCCGTCCTTCA
GCTTCAGCCTCTGCTTGATCTCGCCCTTCAGGGCGCCGTCCTCGGGGTA
CATCCGCTCGGAGGAGGCCTCCCAGCCCATGGTCTTCTTCTGCATTACG
GGGCCGTCGGAGGGGAAGTTGGTGCCGCGCAGCTTCACCTTGTAGATGA
ACTCGCCGTCCTGCAGGGAGGAGTCCTGGGTCACGGTCACCACGCCGCC
GTCCTCGAAGTTCATCACGCGCTCCCACTTGAAGCCCTCGGGGAAGGAC
AGCTTCAAGTAGTCGGGGATGTCGGCGGGGTGCTTCACGTAGGCCTTGG
AGCCGTACATGAACTGAGGGGACAGGATGTCCCAGGCGAAGGGCAGGGG
GCCACCCTTGGTCACCTTCAGCTTGGCGGTCTGGGTGCCCTCGTAGGGG
CGGCCCTCGCCCTCGCCCTCGATCTCGAACTCGTGGCCGTTCACGGAGC
CCTCCATGTGCACCTTGAAGCGCATGAACTCCTTGATGATGGCCATGTT
ATCCTCCTCGCCCTTGCTCACCATGGTGGCGGGATCCGAGCTCGGTACC
AAGCTTGGGTCTCCCTATAGTGAGTCGTATTAATTTCGATAAGCCAGTA
AGCAGTGGGTTCTCTAGTTAGCCAGAGAGCTCTGCTTATATAGACCTCC
CACCGTACACGCCTACCGCCCATTTGCGTCAATGGGGCGGAGTTGTTAC
GACATTTTGGAAAGTCCCGTTGATTTTGGTGCCAAAACAAACTCCCATT
GACGTCAATGGGGTGGAGACTTGGAAATCCCCGTGAGTCAAACCGCTAT
CCACGCCCATTGATGTACTGCCAAAACCGCATCACCATGGTAATAGCGA
TGACTAATACGTAGATGTACTGCCAAGTAGGAAAGTCCCATAAGGTCAT
GTACTGGGCATAATGCCAGGCGGGCCATTTACCGTCATTGACGTCAATA
GGGGGCGTACTTGGCATATGATACACTTGATGTACTGCCAAGTGGGCAG
TTTACCGTAAATAcTCCACCCATTGACGTCAATGGAAAGTCCCTATTGG
CGTTACTATGGGAACATACGTCATTATTGACGTCAATGGGCGGGGGTCG
TTGGGCGGTCAGCCAGGCGGGCCATTTACCGTAAGTTATGTAACGCGGA
ACTCCATATATGGGCTATGAACTAATGACCCCGTAATTGATTACTATTA
ATAACTAGTCAATAATCAATGTCCCAGCTAGCAGAGGGCCTATTTCCCA
TGATTCCTTCATATTTGCATATACGATACAAGGCTGTTAGAGAGATAAT
TGGAATTAATTTGACTGTAAACACAAAGATATTAGTACAAAATACGTGA
CGTAGAAAGTAATAATTTCTTGGGTAGTTTGCAGTTTTAAAATTATGTT
TTAAAATGGACTATCATATGCTTACCGTAACTTGAAAGTATTTCGATTT
CTTGGCTTTATATATCTTGTGGAAAGGACGAAACACCGTTATGGCCGAT
AGGTGCATGTTTTAGAGCTAGAAATAGCAAGTTAAAATAAGGCTAGTCC
GTTATCAACTTGAAAAAGTGGCACCGAGTCGGTGCTTTTTACATCAGGT
TGTTTTTCTGTTTTTACATCAGGTTGTTTTTCTGTTTGGTTTTTTTTTT
ACACCACGTTTATACGCCGGTGCACGGTTTACCACTGAAAACACCTATC
GGCCATAACAGAGACTCTTACATGGTTCCTTTCATACCGCTCTATAGAA
ATGGTGATTTCTTCATAACATCCAAGGATCTGGGATATGACTACAGCTA
CCTCCAAGAGTCAG

### Mouse Care

Mouse stocks used in this study are listed in Table 2. All mice were housed in accordance with federal, state, and IACUC protocols and fed on a standard breeders diet.

TABLE 2
Mouse stocks that were used in this study.

| Jackson | | |
|---|---|---|
| Labs Stock | | |
| Number | Jackson Labs Stock Name | Notes |
| 26175 | B6J.129(B6N)-Gt(ROSA)26Sortm1(CAG-cas9*,-EGFP)Fezh/J | Rosa lox-STOP-lox Cas9 |
| 26179 | B6J.129(Cg)-Gt(ROSA)26Sortm1.1(CAG-cas9*,-EGFP)Fezh/J | Rosa constitutive Cas9 |
| 26816 | B6;129-Igs2tm1(CAG-cas9*)Mmw/J | H11 lox-STOP-lox Cas9 |
| 27650 | STOCK Igs2tm1.1(CAG-cas9*)Mmw/J | H11 constitutive Cas9 |
| 17490 | B6.FVB-Tg(Stra8-icre)1Reb/LguJ | Stra8:Cre |
| 6954 | FVB-Tg(Ddx4-cre)1Dcas/J | Vasa:Cre |
| 4828 | FVB.129P2-Pde6b+ Tyrc-ch/AntJ | Tyrosinase^{Chinchilla} |

### DNA Extraction

<5 mm of tail tissue from each mouse at P21 was obtained. Tail wounds were cauterized with KwikStop Stypic Powder, and screened tails for expression of mCherry using a fluorescent dissecting scope. The tails were submerged in 500 μL of TNES buffer (10 mM Tris, pH 7.5; 400 mM NaCl; 100 mM EDTA; 0.6% SDS) with 3 μL of 10 mg/mL Proteinase K and digested overnight (8-20 hr) in a 56° C. water bath. Then 139 μL of 6M NaCl was added to each sample, vortexed, and centrifuged for 10 minutes at 14,000 g at room temperature. The supernatant was transferred to a clean tube and precipitated DNA by adding 700 μL ice-cold 95% EtOH and samples were placed overnight at −20° C. The precipitated DNA was pelleted by centrifugation at 14,000 g for 10 minutes at 4° C. The pelleted DNA was washed with ice-cold 70% EtOH and allowed it to air-dry before resuspension in TE.

### PCR Reactions

PCR using either Bioline Red MyTaq MasterMix or NEB Q5 2× MasterMix with following recipes and cycling parameters was performed. Where the Bioline Red MyTaq consisted of 1× MasterMix, 0.5 μM primers, 1 μL DNA (between 10-200 ng DNA) in 20 μL with the following cycle parameters, wherein “n” represents the annealing temperature, and “q” represents the elongation time, each is designated in Table 3; (1) 95° C. for 3′; (2) 30 repeats of 95° C. for 15″, n° C. for 15″, 72° C. for q″; (3) 72° C. for 5′; AND (4) 10° C. for ∞.

The NEB Q5 consisted of 1× MasterMix, 0.5 μM primers, 1 μL DNA (between 10-200 ng DNA) in 50 μL and PCR was performed using the following cycle parameters; (1) 98° C. for 30″; (2) 35 repeats of 98° C. for 30″, 64° C. for 30″, 72° C. for 3′; (3) 72° C. for 5′; and (4) 10° C. for Go.

### Gel Purification

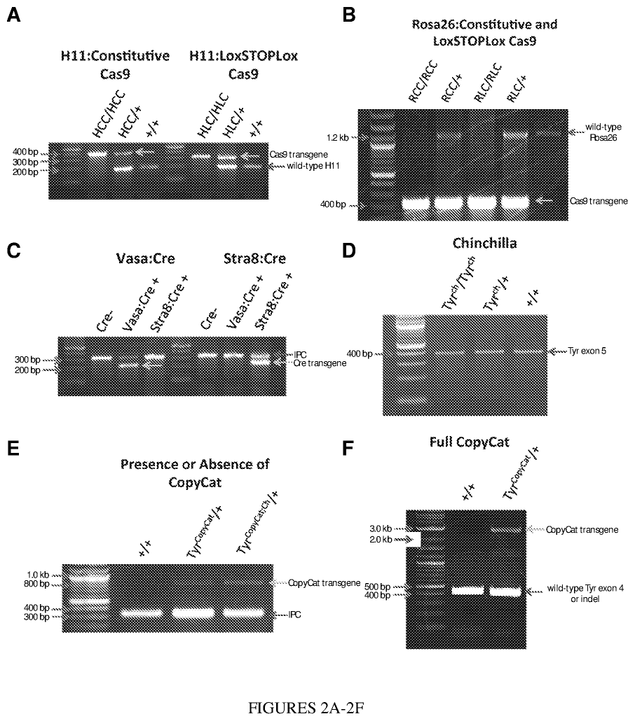

Samples were run on 1-2% agarose gels to separate bands. Samples of each genotyping reaction are in FIG. 2. NHEJ amplicons or Tyr^{Ch} amplicons were gel extracted using a QiaQuick Gel Extraction Kit as instructed. Purified DNA was submitted for Sanger sequencing using the amplification primers noted in the Table 3.

TABLE 3
PCR primers that were used in this study (SEQ ID NOS 8-18, 17, 16, 19-25, 22-23, 26-28, 27,
and 22-23, respectively).

| | | | Ampli- | | Anneal- | Elong- | Sequenc- | |
|---|---|---|---|---|---|---|---|---|
| Primer | | | con | Poly- | ing | ation | ing | |
| Name | Primer Sequence | Amplicon | Length | merase | Temp | Time | Primer | Notes |
| Chin- | GGGAGGAAAGGGTGCTTGAG | Chin- | 392 | MyTaq | 60 | 1′ | Chin- | SNP G > A at |
| chilla | | chilla | bp | | | | chilla | position 252 |
| L3 | | SNP | | | | | R1 | |
| Chin- | CAGCAAGCTGTGGTAGTCGT | | | | | | | |
| chilla | | | | | | | | |
| R1 | | | | | | | | |
| EGFP | ACATGAAGCAGCACGACTTCT | EGFP | 220 | MyTaq | 60 | 45″ | | Indicates |
| L1 | | | bp | | | | | presence of |
| EGFP | ACGTTGTGGCTGTTGTAGTTGT | | | | | | | Rosa26-Cas9 |
| R1 | | | | | | | | and Rosa26- |
| | | | | | | | | loxSTOPloxCas9 |
| RLC- | GTTCGTGCAAGTTGAGTCCATC | RLC-WT | ~1200 | MyTaq | 60 | 45″ | | Indicates |
| WT F* | | | bp | | | | | presence |
| RLC- | GGACTGAGAATAGGCCCAAATG | | | | | | | of Rosa26 |
| WT R* | | | | | | | | wild type |
| | | | | | | | | allele |
| HCC- | GGGCAACGTGCTGGTTATTG | HCC-Tg | 425 | MyTaq | 60 | 10″ | | Indicates |
| Tg-F* | | | bp | | | | | presence |
| HCC- | CCAGGCCGATGCTGTACTTC | | | | | | | of H11-Cas9 |
| Tg-R* | | | | | | | | allele |
| HC- | GGGGCCTCCAAGTCTTGACAGTAGAT | HLC-WT | 200 | MyTaq | 60 | 10″ | | Indicates |
| WT-F* | | | bp | | | | | presence |
| HC- | CTGACCAGTGGGACTGCTTTTTCCAG | | | | | | | of H11 |
| Com- | | | | | | | | wild type |
| mon-R* | | | | | | | | allele |
| HLC- | CGGCCGCCACTCGACGATGTA | HLC-Tg | 350 | MyTaq | 60 | 10″ | | Indicates |
| Cas9- | | | bp | | | | | presence |
| F* | | | | | | | | of H11- |
| HC- | CTGACCAGTGGGACTGCTTTTTCCAG | | | | | | | loxSTOPloxCas9 |
| Com- | | | | | | | | allele |
| mon-R* | | | | | | | | |
| HC- | GGGGCCTCCAAGTCTTGACAGTAGAT | HLC-WT | 200 | MyTaq | 60 | 10″ | | Indicates |
| WT-F* | | | bp | | | | | presence |
| HLC- | CTGACCAGTGGGACTGCTTTTTCCAG | | | | | | | of H11 |
| Com- | | | | | | | | wild type |
| mon-R* | | | | | | | | allele |
| Stra8: | AGATGCCAGGACATCAGGAACCTG | Stra8:Cre | 236 | MyTaq | 60 | 10″ | | Indicates |
| Cre F* | | | bp | | | | | presence |
| Stra8: | ATCAGCCACACCAGACACAGAGATC | | | | | | | of Stra8:Cre |
| Cre R* | | | | | | | | |
| IPC F* | CTAGGCCACAGAATTGAAAGATCT | Internal | 324 | MyTaq | 60 | 10″ | | Internal |
| IPC R* | GTAGGTGGAAATTCTAGCATCATCC | Positive | bp | | | | | Positive |
| | | Control | | | | | | Control |
| | | | | | | | | confirms |
| | | | | | | | | PCR success |
| Vasa: | CACGTGCAGCCGTTTAAGCCGCGT | Vasa:Cre | 240 | MyTaq | 59 | 10″ | | Indicates |
| Cre F* | | | bp | | | | | presence |
| Vasa: | TTCCCATTCTAAACAACACCCTGAA | | | | | | | of Vasa:Cre |
| Cre R* | | | | | | | | |
| IPC F* | CTAGGCCACAGAATTGAAAGATCT | Internal | 324 | MyTaq | 59 | 10″ | | Internal |
| IPC R* | GTAGGTGGAAATTCTAGCATCATCC | Positive | bp | | | | | Positive |
| | | Control | | | | | | Control |
| | | | | | | | | confirms |
| | | | | | | | | PCR success |
| Tyr | AATGGCTGCGAAGGCAC | CopyCat/ | Copy- | Q5 | 64 | 3′ | Tyr HAR | CopyCat band |
| HAL | | NHEJ | Cat: | | | | R2 | indicates |
| F2 | | | 2606 | | | | | presence of |
| Tyr | GGTTCAAAAGCTTCCCAATCCT | | bp | | | | | CopyCat. |
| HAR | | | NHEJ/ | | | | | NHEJ/WT band |
| R2 | | | WT: | | | | | can be |
| | | | ~400 | | | | | sequenced to |
| | | | bp | | | | | identify NHEJ |
| cc F1 | TCAATGTCCCAGCTAGCAGAGGG | CopyCat | 838 | MyTaq | 60 | 1′ | | Indicates |
| Tyr | GGTTCAAAAGCTTCCCAATCCT | | bp | | | | | presence |
| HAR | | | | | | | | of CopyCat |
| R2 | | | | | | | | allele |
| IPC F* | CTAGGCCACAGAATTGAAAGATCT | Internal | 324 | MyTaq | 60 | 1′ | | Internal |
| IPC R* | GTAGGTGGAAATTCTAGCATCATCC | Positive | bp | | | | | Positive |
| | | Control | | | | | | Control |
| | | | | | | | | confirms |
| | | | | | | | | PCR success |

*indicates primers that were designed at the Jackson Laboratory or in respective publications. IPC-internal positive control, amplifies interleukin2 from chromosome 3.

### Example 1

A representative locus was used to assess the feasibility of a CopyCat gene drive that can then be implemented more broadly. The Tyr^{CopyCat} element was inserted into exon 4 of Tyrosinase, the final enzyme of melanin biosynthesis. An sgRNA, designed to target the intact homologous chromosome, was transcribed from a constitutive human U6 promoter. On the reverse strand, mCherry was ubiquitously expressed using the CMV promoter and enhancer. Since the 1.75 kb insert disrupts the Tyr open reading frame, Tyr^{CopyCat} is a functionally null allele.

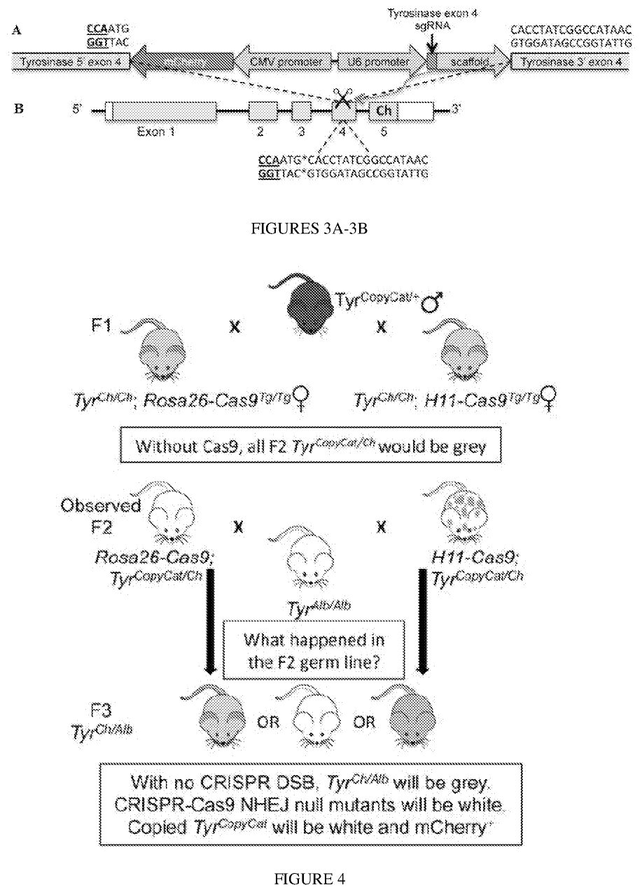

Crossing the Tyr^{CopyCat} mouse to a Cas9 transgenic mouse produced offspring that were heterozygous for both Cas9 and Tyr^{CopyCat} In these mice, the Cas9-sgRNA complex was expected to cleave the intact target site of Tyr exon 4 on the non-transgenic homologous chromosome. The resulting double strand break (asterisks in FIG. 3B) would be repaired either by non-homologous end joining (NHEJ) to produce an indel or by inter-homologue HDR initiated by sequences adjacent to the predicted sgRNA cut site that were identical to genomic sequences flanking the Tyr^{CopyCat} insertion. If inter-homologue HDR repaired the DSB, the Tyr^{CopyCat} allele would be copied to the cleaved chromosome, and the cell would be converted from heterozygous to homozygous Tyr^{CopyCat}. This homozygous Tyr^{CopyCat} provides a gene drive “reporter” mouse (Tyrosinase^{CopyCat}) This mouse encodes an sgRNA in exon 4 of the tyrosinase gene, but unlike the insect Mutagenic Chain Reaction System, it does not also encode the Cas9 gene. The Cas-9 gene drive element is therefore not able to copy itself autonomously and instead requires an exogenous source of Cas9. Consequently, the reporter mouse can be used as a tool to optimize the efficiency of gene drive in different contexts, for example, the reporter mouse can be used to improve the efficiency by altering the developmental timing and cell type specificity of Cas9 expression and by testing modified versions of the Cas9 enzyme.

It has shown that ubiquitous Cas9 expression in the early embryo was able to convert a wild-type allele to Tyrosinase^{CopyCat} by homology directed repair of a CRISPR-Cas9 induced double strand DNA break. These experimental scenarios show the applicability of a CRISPR-Cas9 system in rodents. The system can be applied to a CopyCat element inserted at any locus in the genome. Furthermore, the “cargo” (e.g. insertion of a desired allele) that is encoded together with the sgRNA, here represented by mCherry (FIG. 3A), can be anything that can be genetically encoded. For example, CopyCat alleles can be made that insert exogenous components of a novel biosynthetic pathway into the rodent genome. Resulting engineered rodents may produce compounds not present in wild type animals. Genes from the human genome can also be inserted to replace the homologous rodent counterpart. The humanization of multiple genes in combination may make the rodent a better model and research tool for disease and drug development given recent reports that mouse physiology is in fact quite different from human Genes of the rodent can also be mutated to replicate genetically complex human diseases that require changes at multiple loci. Whereas the combination of a complex set of alleles by Mendelian genetics might make these disease models challenging and expensive to produce, the CopyCat gene drive system will greatly improve the efficiency.

### Example 2

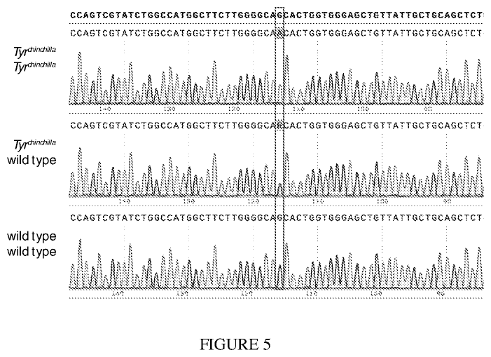

To determine whether a CRISPR-Cas9 gene drive is efficient in the early embryo, the two available “constitutive” Cas9 transgenic lines, Rosa26-Cas9 and H11-Cas9, that reportedly express Cas9 in all organs that have been assessed, were obtained. The Tyr^{Ch} allele was crossed into each of these transgenic lines to genetically mark transmission of the target chromosome and bred both Cas9 and Tyr^{Ch} to homozygosity (FIG. 4). Tyr^{Ch} encodes a hypomorphic point mutation in exon 5, and homozygotes or heterozygotes complemented with a null allele have a grey coat color (8, 9). The G to C single nucleotide polymorphism can also be scored with certainty by PCR followed by DNA sequencing (FIG. 5).

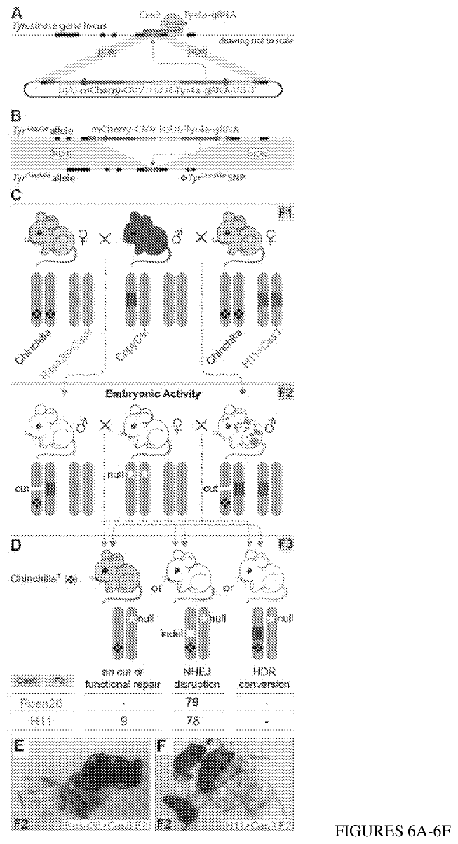

Homozygous female Rosa26-Cas9; Tyr^{Ch/Ch} and H11-Cas9; Tyr^{Ch/Ch} mice were each crossed to Tyr^{CopyCat/+} males with the goal of uniting the paternally transcribed sgRNA and maternally provided Cas9 protein in the early embryo (FIG. 3C). In absence of a second loss-of-function mutation in exon 4 of the target chromosome, Tyr^{CopyCat/Ch} mice should appear grey (Tyr^{CopyCat/Ch}; Cas9− mice in FIG. 6E). However, no grey Tyr^{CopyCat/Ch}; Cas9+ mice in the F2 offspring of either cross were observed. Instead, 94% of Rosa26-Cas9; Tyr^{CopyCat/Ch} mice were entirely white (17 white: 1 mosaic), while 87.5% of H11-20 Cas9; Tyr^{CopyCat/Ch} mice were a mosaic mixture of grey and white fur (21 mosaic: 3 white) (FIGS. 6E and 6F and Table 4).

TABLE 4
Coat color of F2 individuals that were
constitutive Cas9+ and Tyr^{CopyCat/Chinchilla}.

| | Rosa26 > Cas9 | H11 > Cas9 |
|---|---|---|
| White | 17 | 3 |
| Mosaic | 1 | 21 |

These results illustrate the highly efficient action of the Tyr gRNA, as well as a qualitative difference in the efficiency and/or timing of Cas9 activity driven by the Rosa26-Cas9 and the H11-Cas9 transgenes.

In order to assess the efficiency of copying the Tyr^{CopyCat} allele to the target chromosome and subsequent transmission to the next generation, these mice were crossed to mice that were homozygous for a null Tyrosinase mutation in exon 1. Without gene drive, mice that inherit the Tyr^{Ch} allele together with a null allele will be grey. Animals that genotype for the Tyr^{Ch} allele but that are white and fluoresce red indicate successful CRISPR-Cas9 mediated copying of the Tyr^{CopyCat} allele into the intact exon 4. These mice are white due to inheritance of two null alleles, and they fluoresce red due to the mCherry cargo gene in the gene drive element that was copied to the Tyr^{Ch} marked chromosome. The F3 offspring of five H11-Cas9 lineage males were assessed, and one gene drive copying event out of 79 Tyr^{Ch} individuals (1.3% efficiency) was observed. One copying event out of a total of 64 offspring derived from four males in the Rosa26-Cas9 lineage (1.6% efficiency) was also observed. The low rate of copying and transmission suggests zygotic/embryonic Cas9 expression is insufficient and instead indicates germline restriction of Cas9 may be crucial. The copying events are however evidence that the gene drive reporter mouse works as designed and is a valuable resource to optimize the gene drive system in rodents.

The high rate of mutagenesis in the Rosa26-Cas9 lineage is of extraordinary research value. 100% of the 64 F3 offspring of this lineage were white mice. If other sgRNAs cut their target sites with similar efficiency, the present invention can be used to simultaneously and efficiently knock out the function of multiple genes.

In the absence of gene conversion, the Tyr^{CopyCat} allele would be expected to transmit by Mendelian inheritance to 50% of the progeny of an outcross. In such cases, effectively none of the Tyr^{Ch}-marked target chromosomes would be expected to carry the Tyr^{CopyCat} allele due to ultra-tight linkage of Tyr exons 4 and 5, which are separated by only ˜9 kb. In order to assess inheritance in a large number of offspring, each F2 male Rosa26-Cas9; Tyr^{CopyCat/Ch} and H11-Cas9; Tyr^{CopyCat/Ch} mouse was crossed to multiple albino CD-1 females (Tyr^{Null}), which carry a loss-of-function point mutation in Tyr exon 1 (5, 9) (FIG. 6D). The F3 offspring of this cross were genotyped by PCR and DNA sequencing to identify offspring that inherited the Tyr^{Ch}-marked target chromosome.

In absence of a second null mutation in exon 4 of the Tyr^{Ch}-marked chromosome, Tyr^{Ch/Null} animals should appear grey due to partial activity of the hypomorphic Tyr^{Ch} allele. However, all F3 offspring of the Rosa26-Cas9 lineage and 89.7% of F3 offspring of the H11-Cas9 lineage were white, indicating that transmission of CRISPR/Cas9 induced loss-of-function mutations on the Tyr^{Ch}-marked chromosome is consistent with F2 coat color mosaicism of the parents (FIGS. 6D, 6E, and 6F and Table 4 and 5).

TABLE 5
Phenotypes of the F3 progeny of a subset of individuals listed in Table 1.

| | | | Chinchilla + F3 offspring | | | |
|---|---|---|---|---|---|---|
| | | | | | Grey | |
| | | | | White | (no cut or | mCherry + |
| | | Chinchilla − | Chinchilla + | (NHEJ | functional | (HDR |
| | F2 parent color | Total F3 | Total F3 | mutation) | repair) | conversion) |
| Rosa26 Family 1 | white | 19 | 10 | 10 | 0 | 0 |
| Rosa26 Family 2 | white | 11 | 15 | 15 | 0 | 0 |
| Rosa26 Family 3* | white | 27* | 16 | 16 | 0 | 0 |
| Rosa26 Family 4 | white | 22 | 22 | 22 | 0 | 0 |
| Rosa26 Family 5 | white | 16 | 16 | 16 | 0 | 0 |
| H11 Family 1 | prim. white mosaic | 14 | 15 | 15 | 0 | 0 |
| H11 Family 2 | prim. grey mosaic | 25 | 31 | 28 | 3 | 0 |
| H11 Family 3 | prim. white mosaic | 8 | 2 | 2 | 0 | 0 |
| H11 Family 4 | prim. white mosaic | 12 | 15 | 15 | 0 | 0 |
| H11 Family 5 | mosaic | 23 | 24 | 18 | 6 | 0 |

*indicates a family with a possible large deletion that encompasses the Tyr^{Chinchilla} SNP

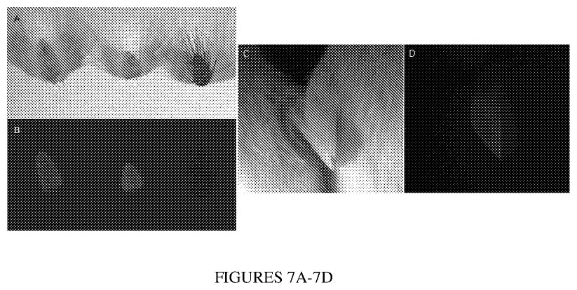

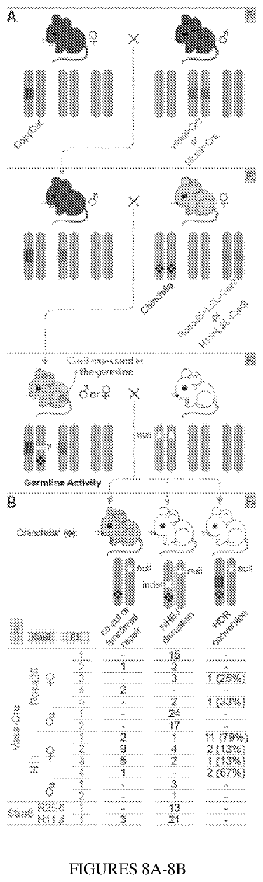

If the induced null alleles resulted from interhomologue HDR to copy the Tyr^{CopyCat} allele to the Tyr^{Ch}-marked target chromosome, these white animals should also express the fluorescent mCherry marker. However, none of the F3 offspring that inherited the Tyr^{Ch}-marked target chromosome in either the Rosa26- or H11-Cas9 lineages expressed mCherry. Consistent with the lack of mCherry expression, PCR amplification of Tyr exon 4 revealed NHEJ-induced indels in white progeny (FIG. 6D and FIG. 7).

The different propensities to yield full albino versus mosaic coat color patterns in the Rosa26-Cas9 and H11-Cas9 lineages were also paralleled by differences in the number of unique NHEJ mutations in individuals of each genotype. Sequenced PCR products from Rosa26-Cas9; Tyr^{CopyCat} F2 tails (somatic tissues that are comprised of ectodermal and mesodermal derivatives) and from individual F3 outcross offspring (representing the germline) routinely exhibited only two unique NHEJ mutations suggesting that many of these Cas9 induced mutations were generated in 2-4 cell stage embryos. In contrast, H11-Cas9; Tyr^{CopyCat} F2 tails and F3 offspring harbored several different NHEJ mutations, which suggests that Cas9 is active at later embryonic stages and/or at lower levels in this lineage.

The formation of indels in the early embryo provides an efficient method to generate mutations in a given gene with a low level of mosaicism that would produce predictable whole organism phenotypes. Since such mutations are generated with high efficiency using Rosa26-Cas9 transgenic mice, it should be possible to design an active genetic element encoding several gRNAs that target multiple genes simultaneously to evaluate the consequence of combinatorial gene knock-outs in a simple heritable system. These results are also relevant to recent reports showing that early zygotic CRISPR/Cas9 induced DSBs are repaired by inter-homologue HDR in mouse and human embryos (3, 4). The presence of so few unique NHEJ mutations in the Rosa26-Cas9 lineage suggests that zygotic inter-homologue HDR is transiently limited to a window of time very near fertilization.

Two reasons for the absence of Tyr^{CopyCat} copying to the target chromosome in the early embryo were considered. The first possibility was, that homologous chromosomes are not aligned to allow for efficient strand invasion that is necessary for inter-homologue HDR to repair DSBs. Alternatively, the DNA repair machinery that is active in somatic cells typically favors NHEJ over HDR, which would generate indels that obliterate the gRNA cut site in the early embryo. A solution to overcome these two potential obstacles is to restrict CRISPR/Cas9 activity to occur during meiosis in the developing germline One purpose of sexual reproduction is to “shuffle the deck” by recombining the maternal and paternal genomes at each generation. Meiotic recombination is initiated by the intentional formation of DSBs that are repaired by exchange of DNA sequence information between homologous chromosomes that are physically paired during Meiosis I (10). Indeed, the molecular mechanisms of NHEJ are repressed during meiosis in many species, including mice (11), likely because activity of the NHEJ pathway in the germline would be highly mutagenic (12).

In order to test the whether Cas9 activity during meiotic recombination will convert a heterozygous active genetic element to homozygosity, we designed a crossing scheme to introduce the first expression of Cas9 in the presence of the Tyr^{CopyCat} allele during germline development. Since there currently are no available transgenic mice that express Cas9 under direct control of a germline-specific promoter, conditional Rosa26- or H11-LSLCas9 transgenes were combined, each with a Lox-Stop-Lox preceding the Cas9 translation start site (6, 7), with available Vasa-Cre or Stra8-Cre germline transgenic mice. Vasa-Cre is expressed later than the endogenous Vasa transcript in both male and female germ cells (13) while Stra8-Cre expression is limited to the male germline and is initiated in early stage spermatogonia (14). Although oogonia and spermatogonia are pre-meiotic, and spermatogonia are in fact mitotic, reasoning that Cre protein must first accumulate to recombine the conditional Cas9 allele for subsequent Cas9 protein expression and activity. The time delay may require initiation of Cre expression prior to the onset of meiosis so that DSBs are resolved by inter-homologue HDR prior to segregation of homologous chromosomes at the end of Meiosis I. Each combination of these Cre and conditional Cas9 lines was created in case the timing or levels of Cas9 expression are critical variables in these crosses. Males and females of the Vasa strategies were also assessed in case there are sex-dependent differences in animals that inherit the same genotype.

Males heterozygous for Tyr^{CopyCat} and the Vasa-Cre transgene were crossed to females homozygous for both the Tyr^{Ch} allele and one of the two conditional Cas9 transgenes (FIG. 8A). Reasoning that in the reverse cross (i.e., using female Vasa-Cre mice), Cre protein that is maternally deposited in the egg might prematurely induce recombination of the conditional Cas9 allele (13). Early embryonic Cas9 expression would have led to somatic mutagenesis similar to that observed in the experiments above using constitutive Cas9 transgenes. Instead, introducing the Vasa-Cre transgene by inheritance from the male resulted in most offspring that were entirely grey, due to the Tyr^{CopyCat/Ch} genotype, and a few mosaic animals (Table 6). The presence of any mosaicism suggests that even this approach for conditional germline restricted Cas9 expression resulted in some degree of leakiness in somatic tissues.

TABLE 6
Coat color of Tyr^{CopyCat/Chinchilla} F3 individuals
that inherited a germline Cre transgene and
a loxSTOPlox: Cas9 conditional allele.

| | Vasa > Cre | | | | Stra8 > Cre | |
|---|---|---|---|---|---|---|
| | Rosa26 > | | H11 > | | Rosa26 > | H11 > |
| | LSLCas9 | | LSLCas9 | | LSLCas9 | LSLCas9 |
| | Female | Male | Female | Male | Male | Male |
| White | 0 | 0 | 0 | 0 | 0 | 0 |
| Grey | 3 | 4 | 5 | 4 | 2 | 1 |
| Mosaic | 2 | 1 | 0 | 1 | 0 | 0 |

Whether expression of CRISPR/Cas9 in the female germline could promote copying of the Tyr^{CopyCat} element onto the target chromosome by crossing female mice of each Vasa-Cre lineage to CD-1 (Tyr^{Null}) males was tested. In each cross, offspring that inherited the Tyr^{Ch}-marked chromosome was identified. As in the cross to assess the effects of embryonic Cas9 expression above, we expected Tyr^{Ch/Null} mice without a second loss-of-function mutation in exon 4 of the target chromosome would be grey. Mice with a CRISPR/Cas9 induced NHEJ mutation in exon 4 would be expected to appear white. Mice with a CRISPR/Cas9 induced mutation that was repaired by inter-homologue HDR should also be white but additionally fluoresce red due to transmission of the mCherry-marked Tyr^{CopyCat} active genetic element (FIG. 8B).

FIG. 8B summarizes the results of these crosses to test the effects of Cas9 activity in the female germline. In contrast with constitutive embryonic expression of Cas9, it was observed that the Tyr^{CopyCat} transgene was copied to the Tyr^{Ch}-marked target chromosome in both Vasa-Cre; Rosa26-LSL-Cas9 and Vasa-Cre; H11-LSL-Cas9 lineages. However, the efficiency differed between genotypes and also between females with the same genotype (Table 7).

TABLE 7
Phenotypes of the Tyr^{Chinchilla+} F4 offspring of F3 individuals listed in Table 6. The second
and third columns in each panel designate the total number of
Tyr^{Chinchilla-} and Tyr^{Chincilla+} offspfing
Vasa > Cre
Rosa26 > LSL Cas9
Female

| | | | Chinchilla + F4 offspring | | | | | |
|---|---|---|---|---|---|---|---|---|
| | | | | White, | | Grey | White, | |
| | | | | mCherry | Mosaic | (no cut | mCherry | |
| | | Chinchilla | Chinchilla | − | (variable | or | + | % |
| | F3 | − | + | (NHEJ | NHEJ | func- | (HDR | Copying |
| | parent | F4 | F4 | muta- | muta- | tional | con- | in F3 |
| | color | Total | Total | tion) | tions) | repair) | version) | germline |
| Rosa26 Family 1 | mosaic | 10 | 15 | 14 | 1 | 0 | 0 | 0.0 |
| Rosa26 Family 2 | grey | 8 | 3 | 1 | 1 | 1 | 0 | 0.0 |
| Rosa26 Family 3* | mosaic | 6 | 4 | 3 | 0 | 0 | 1 | 25.0 |
| Rosa26 Family 4 | grey | 1 | 2 | 0 | 0 | 2 | 0 | 0.0 |
| Rosa26 Family 5 | grey | 3 | 3 | 0 | 2 | 0 | 1 | 33.3 |
| H11 Family 1 | grey | 10 | 14 | 0 | 1 | 2 | 11 | 78.6 |
| H11 Family 2 | grey | 15 | 15 | 2 | 2 | 9 | 2 | 13.3 |
| H11 Family 3 | grey | 2 | 8 | 1 | 1 | 5 | 1 | 12.5 |
| H11 Family 4 | grey | 2 | 3 | 0 | 0 | 1 | 2 | 66.7 |

Vasa > Cre
Rosa26 > LSL Cas9
Male

| | | | Chinchilla + F4 offspring | | | | | |
|---|---|---|---|---|---|---|---|---|
| | | | | White, | | Grey | White, | |
| | | | | mCherry | Mosaic | (no cut | mCherry | |
| | | Chinchilla | Chinchilla | − | (variable | or | + | % |
| | F3 | − | + | (NHEJ | NHEJ | func- | (HDR | Copying |
| | parent | F4 | F4 | muta- | muta- | tional | con- | in F3 |
| | color | Total | Total | tion) | tions) | repair) | version) | germline |
| Rosa26 Family 1 | grey | 29 | 24 | 24 | 0 | 0 | 0 | 0.0 |
| Rosa26 Family 2 | grey | 19 | 17 | 17 | 0 | 0 | 0 | 0.0 |
| H11 Family 1 | grey | 11 | 3 | 3 | 0 | 0 | 0 | 0.0 |
| H11 Family 2 | grey | 3 | 1 | 1 | 0 | 0 | 0 | 0.0 |

Stra8 > Cre
Rosa26 > LSL Cas9
Male

| | | | Chinchilla + F4 offspring | | | | | |
|---|---|---|---|---|---|---|---|---|
| | | | | White, | | Grey | White, | |
| | | | | mCherry | Mosaic | (no cut | mCherry | |
| | | Chinchilla | Chinchilla | − | (variable | or | + | % |
| | F3 | − | + | (NHEJ | NHEJ | func- | (HDR | Copying |
| | parent | F4 | F4 | muta- | muta- | tional | con- | in F3 |
| | color | Total | Total | tion) | tions) | repair) | version) | germline |
| Rosa26 Family 1 | grey | 14 | 13 | 13 | 0 | 0 | 0 | 0.0 |

Str8 > Cre
H11 > LSL Cas9
Male

| | | | Chinchilla + F4 offspring | | | | | |
|---|---|---|---|---|---|---|---|---|
| | | | | White, | | Grey | White, | |
| | | | | mCherry | Mosaic | (no cut | mCherry | |
| | | Chinchilla | Chinchilla | − | (variable | or | + | % |
| | F3 | − | + | (NHEJ | NHEJ | func- | (HDR | Copying |
| | parent | F4 | F4 | muta- | muta- | tional | con- | in F3 |
| | color | Total | Total | tion) | tions) | repair) | version) | germline |
| H11 Family 1 | grey | 22 | 24 | 21 | 0 | 3 | 0 | 0.0 |

Specifically, two out of five females of the Vasa-Cre; Rosa26-LSL-Cas9 lineage and all four of four females of the Vasa-Cre; H11-LSL-Cas9 lineage transmitted a chromosome containing both Tyr^{CopyCat} and Tyr^{Ch}, the product of inter-homologue HDR, to at least one offspring. The highest efficiency of genotype conversion within a germline produced 11 out of 14 offspring (78.6%) with a Tyr^{CopyCat} insertion on the Tyr^{Ch}-marked chromosome in the Vasa-Cre; H11-LSL-Cas9 lineage (FIG. 8B and Table 7). These data demonstrate transmission of the Tyr^{CopyCat} element that was copied onto the Tyr^{Ch}-marked chromosome, an event with a very low probability (4.7×10⁻⁵) by natural mechanisms due to linkage. In contrast, copying of the Tyr^{CopyCat} element was not observed in crosses where conditional Cas9 expression was induced by Vasa-Cre in males. Consistent with this finding, Tyr^{CopyCat} was not copied to the Tyr^{Ch}-marked target chromosome in the male germline of Stra8-Cre lineages (FIG. 8B and Table 7).

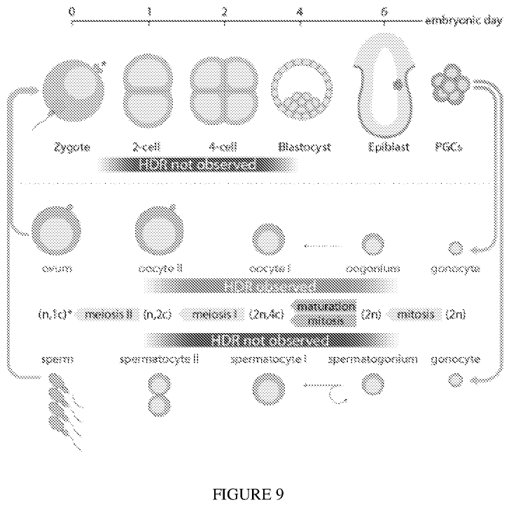

In mammals, spermatogonia continually undergo mitosis throughout the life of the male to produce new primary spermatocytes (15). It is therefore possible that even the delayed Cre dependent strategy induced DSBs in mitotic spermatocytes that were repaired by NHEJ, and the cut site was mutated prior to the onset of meiosis. In contrast, oogonia directly enlarge without further mitosis to form all of the primary oocytes (16). These arrest during embryogenesis, prior to the first meiotic division, and oocyte maturation and meiosis continues after puberty. The higher efficiency of inter-homologue HDR in females of the H11-LSL-Cas9 conditional strategy may reflect lower or delayed Cas9 expression from the H11 locus compared to Rosa26, also evident from a comparison of coat colors in the constitutive crosses. Thus, in the Vasa-Cre; H11-LSL-Cas9 mice, Cas9 activity may have been fortuitously delayed to fall within an optimal window during female meiosis. The observed difference in the efficiency of inter-homologue HDR between females and males and even among females therefore likely indicates a requirement for the precise timing of CRISPR/Cas9 activity; NHEJ indels might reflect DSB repair that occurred prior to alignment of homologous chromosomes during Meiosis I or after their segregation (FIG. 9). Together, these results demonstrate that highly efficient active genetic approaches are feasible in mammals and open the door to further optimization and implementation of genetic schemes that leverage super-Mendelian inheritance to assemble complex genotypes, including combinations of closely linked genes.

As noted above, regardless of whether homology directed repair copies the element as designed, the utility of the present system has been demonstrated to produce zygotic null mutations at high frequency. It is therefore possible to encode multiple sgRNAs at a single locus that will combine with Cas9 protein to cut their target sites throughout the genome. The resulting animals would be compound homozygous knockout for each target gene derived from a single insertion. This approach would substantially increase the efficiency and decrease the cost of producing multiple targeted alleles of redundant genes. Furthermore, many human diseases, syndromes, and disorders are the effect of chromosomal deletions or translocations that eliminate function of multiple genes (e.g. Williams-Beuren Syndrome, which deletes approximately 28 genes). By encoding clusters of sgRNAs that target subsets of these genes, it will be possible to multiplex compound knockout alleles to assess multigenic phenotypes, and the present method can be implemented in a wide variety of applications to model and research diseases, test drug efficacy, and metabolism.

### Example 3

A CRISPR-Cas9 gene drive system stands to revolutionize rodent breeding. If each desired allele is encoded as a gene drive element that contains an sgRNA designed to target the same genomic location in the wild type homologous chromosome, each locus will be “driven” to homozygosity in the presence of Cas9. Therefore, in order to combine three alleles, for example, a mouse with one gene drive element (A) would be crossed to a mouse that encodes Cas9. Offspring of this cross would then be crossed to mice carrying gene drive element B, and these offspring would be crossed to mice carrying gene drive element C. In the presence of Cas9 at each generation, these gene drive elements at three distinct loci will be converted to homozygosity such that 50% of offspring, those that inherit Cas9, will be triple homozygous after three generations, even if they are genetically linked loci.

### REFERENCES

1. Gantz, V. M. & Bier, E. The mutagenic chain reaction: A method for converting heterozygous to homozygous mutations. Science 348, 442-444 (2015).
2. Gantz, V. M., Jasinskiene, N., Tatarenkova, O., Fazekas, A., Macias, V. M., Bier, E. & James, A. A. Highly efficient Cas9-mediated gene drive for population modification of the malaria vector mosquito Anopheles stephensi. PNAS 112, E6736-E6743 (2015).
3. Y. Wu et al., Correction of a genetic disease in mouse via use of CRISPR-Cas9. Cell Stem Cell. 13, 659-662 (2013).
4. H. Ma et al., Correction of a pathogenic gene mutation in human embryos. Nature. 548, 413-419 (2017).
5. T. Yokoyama et al., Conserved cysteine to serine mutation in tyrosinase is responsible for the classical albino mutation in laboratory mice. Nucleic Acids Res. 18, 7293-7298 (1990).
6. R. J. Platt et al., CRISPR-Cas9 knockin mice for genome editing and cancer modeling. Cell. 159, 440-455 (2014).
7. S.-H. Chiou et al., Pancreatic cancer modeling using retrograde viral vector delivery and in vivo CRISPR/Cas9-mediated somatic genome editing. Genes Dev. 29, 1576-1585 (2015).
8. H. W. Feldman, A Fourth Allelomorph in the Albino Series in Mice. Am. Nat. 56, 573-574 (1922).
9. F. Beermann et al., Rescue of the albino phenotype by introduction of a functional tyrosinase gene into mice. EMBO J. 9, 2819-2826 (1990).
10. S. Keeney, C. N. Giroux, N. Kleckner, Meiosis-specific DNA double-strand breaks are catalyzed by Spo11, a member of a widely conserved protein family. Cell. 88, 375-384 (1997).
11. W. Goedecke, M. Eijpe, H. H. Offenberg, M. van Aalderen, C. Heyting, Mre11 and Ku70 interact in somatic cells, but are differentially expressed in early meiosis. Nat. Genet. 23, 194-198 (1999).
12. E. F. Joyce, A. Paul, K. E. Chen, N. Tanneti, K. S. McKim, Multiple barriers to nonhomologous DNA end joining during meiosis in Drosophila. Genetics. 191, 739-746 (2012).
13. T. Gallardo, L. Shirley, G. B. John, D. H. Castrillon, Generation of a germ cell-specific mouse transgenic Cre line, Vasa-Cre. Genesis. 45, 413-4-17 (2007).
14. P. I. Sadate-Ngatchou, C. J. Payne, A. T. Dearth, R. E. Braun, Cre recombinase activity specific to postnatal, premeiotic male germ cells in transgenic mice. Genesis. 46, 738-742 (2008).
15. D. G. de Rooij, J. A. Grootegoed, Spermatogonial stem cells. Curr. Opin. Cell Biol. 10, 694-701 (1998).
16. M. E. Pepling, From primordial germ cell to primordial follicle: mammalian female germ Cell development. Genesis. 44, 622-632 (2006).

1. A method of producing a genetically modified rodent comprising introducing into the rodent a Cas9-mediated split gene-drive system to actively edit a genome sequence of the rodent.

2. The method of claim 1, wherein the Cas9-mediated split gene-drive system is introduced by crossing a strain A carrying a genetic element A with a strain B, carrying a genetic element B, wherein strain A does not have a desired allele at an endogenous location, and wherein the genetic element A comprises the desired allele.

3. The method of claim 2, wherein the genetic element A encodes: 1) a gRNA driving the genetic element A at its insertion site, 2) a gRNA driving the genetic element B at its insertion site, and 3) multiple gRNAs targeting coding sequences of several genes to multiplex compound desired alleles required for assessing mutagenic phenotypes.

4. The method of claim 3, wherein the genetic element B encodes a Cas9 endonuclease.

5. The method of claim 4, wherein when rodents of strain A are crossed to rodents of strain B, an expressed Cas9 endonuclease encoded by element B drives copying of the genetic elements A and B onto identical insertion sites at their respective sister chromosomes and sequence mutations at sites targeted by the multiple gRNAs targeting coding sequences of several genes required for the desired transgenic rodent.

6. The method of claim 5, wherein progeny carrying both of the genetic elements A and B contain the desired alleles, and transmit the desired alleles on to 95% or more of the progeny's progeny and subsequent generations.

7. The method of claim 2, wherein progeny resulting from crossing the strain A with the strain B are sterile rodents.

8. The method of claim 2, wherein the genetic element B encodes a Cas9 endonuclease and a gRNA driving the genetic element B at its insertion site, thereby creating a full gene drive at the locus for genetic element B.

9. The method of claim 2, wherein the genetic elements A and B or corresponding genomic insertion sites on wild-type chromosomes further comprise a fluorescent marker gene to distinguish transgenic from wild-type chromosomes.

10. The method of claim 1, wherein the activity of the Cas9-mediated split gene-drive system is restricted to occur during meiosis.

11. The method of claim 10, wherein the activity is further restricted to meiosis I.

12. A genetically modified reporter animal having the sgRNA driving element of a Cas9-mediated split gene-drive system for mutating rodents.

13. A genetically modified rodent having a Cas9-mediated split gene-drive system for mutating rodents, wherein said mutation may include individually or any combination of a deletion, a translocation, or an addition.

14. The genetically modified rodent of claim 13, wherein the Cas9-mediated split gene-drive system causes mutations to fertility loci.

15. The genetically modified rodent of claim 13, wherein the Cas9-mediated split gene-drive system causes mutations to pesticide-resistance loci.

16. The genetically modified rodent of claim 13, wherein the Cas9-mediated split gene-drive system includes addition of at least one desired allele.

17. The genetically modified rodent of claim 13, wherein the Cas9-mediated split gene-drive system comprises a first genetic element and a second genetic element.

18. A method of suppressing a wild-type population of rodents comprising breeding the wild-type population with a genetically modified rodent population having a Cas9-mediated split gene-drive system of claim 14.

19. A method of suppressing a wild-type population of rodents comprising breeding the wild-type population with a genetically modified rodent population having a Cas9-mediated split gene-drive system of claim 15.


SEQUENCE LISTING (33 sequences)

SEQ ID NO: 1 - DNA, 25 bp, Artificial Sequence
  Description of Artificial Sequence Synthetic primer
ccagctagcagagggcctatttccc

SEQ ID NO: 2 - DNA, 22 bp, Artificial Sequence
  Description of Artificial Sequence Synthetic primer
gagctcgaattcactggccgtc

SEQ ID NO: 3 - DNA, 60 bp, Artificial Sequence
  Description of Artificial Sequence Synthetic primer
taggccctctgctagctgggacattgattattgactagttattaatagtaatcaattacg

SEQ ID NO: 4 - DNA, 48 bp, Artificial Sequence
  Description of Artificial Sequence Synthetic primer
tgtaaaacgacggccagtgaattcgagctcccatagagcccaccgcat

SEQ ID NO: 5 - DNA, 54 bp, Artificial Sequence
  Description of Artificial Sequence Synthetic primer
gttatggccgataggtgcatgttttagagctagaaatagcaagttaaaataagg

SEQ ID NO: 6 - DNA, 22 bp, Artificial Sequence
  Description of Artificial Sequence Synthetic primer
ggtgtttcgtcctttccacaag

SEQ ID NO: 7 - DNA, 2366 bp, Artificial Sequence
  Description of Artificial Sequence Synthetic polynucleotide
tatttttgaacaatggctgcgaaggcaccgccctcttttggaagtttacccagaagccaatgccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgagctctagcatttaggtgacactatagaatagggccctctagatgcatgctcgagcggccgccagtgtgatggatatctgcagaattcttacttgtacagctcgtccatgccgccggtggagtggcggccctcggcgcgttcgtactgttccacgatggtgtagtcctcgttgtgggaggtgatgtccaacttgatgttgacgttgtaggcgccgggcagctgcacgggcttcttggccttgtaggtggtcttgacctcagcgtcgtagtggccgccgtccttcagcttcagcctctgcttgatctcgcccttcagggcgccgtcctcggggtacatccgctcggaggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtacatgaactgaggggacaggatgtcccaggcgaagggcagggggccacccttggtcaccttcagcttggcggtctgggtgccctcgtaggggcggccctcgccctcgccctcgatctcgaactcgtggccgttcacggagccctccatgtgcaccttgaagcgcatgaactccttgatgatggccatgttatcctcctcgcccttgctcaccatggtggcgggatccgagctcggtaccaagcttgggtctccctatagtgagtcgtattaatttcgataagccagtaagcagtgggttctctagttagccagagagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcccagctagcagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgttatggccgataggtgcatgttttagagctagaaatagcaagttaaaataaggctagtccgttatcaacttgaaaaagtggcaccgagtcggtgctttttacatcaggttgtttttctgtttttacatcaggttgtttttctgtttggttttttttttacaccacgtttatacgccggtgcacggtttaccactgaaaacacctatcggccataacagagactcttacatggttcctttcataccgctctatagaaatggtgatttcttcataacatccaaggatctgggatatgactacagctacctccaagagtcag

SEQ ID NO: 8 - DNA, 20 bp, Artificial Sequence
  Description of Artificial Sequence Synthetic primer
gggaggaaagggtgcttgag

SEQ ID NO: 9 - DNA, 20 bp, Artificial Sequence
  Description of Artificial Sequence Synthetic primer
cagcaagctgtggtagtcgt

SEQ ID NO: 10 - DNA, 21 bp, Artificial Sequence
  Description of Artificial Sequence Synthetic primer
acatgaagcagcacgacttct

SEQ ID NO: 11 - DNA, 22 bp, Artificial Sequence
  Description of Artificial Sequence Synthetic primer
acgttgtggctgttgtagttgt

SEQ ID NO: 12 - DNA, 22 bp, Artificial Sequence
  Description of Artificial Sequence Synthetic primer
gttcgtgcaagttgagtccatc

SEQ ID NO: 13 - DNA, 22 bp, Artificial Sequence
  Description of Artificial Sequence Synthetic primer
ggactgagaataggcccaaatg

SEQ ID NO: 14 - DNA, 20 bp, Artificial Sequence
  Description of Artificial Sequence Synthetic primer
gggcaacgtgctggttattg

SEQ ID NO: 15 - DNA, 20 bp, Artificial Sequence
  Description of Artificial Sequence Synthetic primer
ccaggccgatgctgtacttc

SEQ ID NO: 16 - DNA, 26 bp, Artificial Sequence
  Description of Artificial Sequence Synthetic primer
ggggcctccaagtcttgacagtagat

SEQ ID NO: 17 - DNA, 26 bp, Artificial Sequence
  Description of Artificial Sequence Synthetic primer
ctgaccagtgggactgctttttccag

SEQ ID NO: 18 - DNA, 21 bp, Artificial Sequence
  Description of Artificial Sequence Synthetic primer
cggccgccactcgacgatgta

SEQ ID NO: 19 - DNA, 26 bp, Artificial Sequence
  Description of Artificial Sequence Synthetic primer
ctgaccagtgggactgctttttccag

SEQ ID NO: 20 - DNA, 24 bp, Artificial Sequence
  Description of Artificial Sequence Synthetic primer
agatgccaggacatcaggaacctg

SEQ ID NO: 21 - DNA, 25 bp, Artificial Sequence
  Description of Artificial Sequence Synthetic primer
atcagccacaccagacacagagatc

SEQ ID NO: 22 - DNA, 24 bp, Artificial Sequence
  Description of Artificial Sequence Synthetic primer
ctaggccacagaattgaaagatct

SEQ ID NO: 23 - DNA, 25 bp, Artificial Sequence
  Description of Artificial Sequence Synthetic primer
gtaggtggaaattctagcatcatcc

SEQ ID NO: 24 - DNA, 24 bp, Artificial Sequence
  Description of Artificial Sequence Synthetic primer
cacgtgcagccgtttaagccgcgt

SEQ ID NO: 25 - DNA, 25 bp, Artificial Sequence
  Description of Artificial Sequence Synthetic primer
ttcccattctaaacaacaccctgaa

SEQ ID NO: 26 - DNA, 17 bp, Artificial Sequence
  Description of Artificial Sequence Synthetic primer
aatggctgcgaaggcac

SEQ ID NO: 27 - DNA, 22 bp, Artificial Sequence
  Description of Artificial Sequence Synthetic primer
ggttcaaaagcttcccaatcct

SEQ ID NO: 28 - DNA, 23 bp, Artificial Sequence
  Description of Artificial Sequence Synthetic primer
tcaatgtcccagctagcagaggg

SEQ ID NO: 29 - DNA, 17 bp, Mus musculus
cacctatcggccataac

SEQ ID NO: 30 - DNA, 23 bp, Mus musculus
ccaatgcacctatcggccataac

SEQ ID NO: 31 - DNA, 64 bp, Mus musculus
ccagtcgtatctggccatggcttcttggggcagcactggtgggagctgttattgctgcagctct

SEQ ID NO: 32 - DNA, 64 bp, Mus musculus
ccagtcgtatctggccatggcttcttggggcaacactggtgggagctgttattgctgcagctct

SEQ ID NO: 33 - DNA, 64 bp, Mus musculus
ccagtcgtatctggccatggcttcttggggcarcactggtgggagctgttattgctgcagctct